(19) United States
(12) Patent Application Publication
(10) Pub. No.: US 2024/0336879 A1
(43) Pub. Date: Oct. 10, 2024

(54) IMPROVEMENTS IN OR RELATING TO ORGANIC COMPOUNDS

(71) Applicant: Givaudan SA, VERNIER (CH)

(72) Inventor: Antoine CHARBONNIER, Argenteuil (FR)

(21) Appl. No.: 18/293,739

(22) PCT Filed: Aug. 9, 2022
(86) PCT No.: PCT/EP2022/072304

§ 371 Date: Jan. 30, 2024

(30) Foreign Application Priority Data
Aug. 16, 2021 (GB) 2111712.2

(51) Int. Cl. C11D 3/50; B01J 13/10; C11D 3/37; C11D 17/00

(52) U.S. Cl. CPC C11D 3/505; B01J 13/10; C11D 3/3742; C11D 17/0039

(57) ABSTRACT

Described is an encapsulated composition comprising at least one core-shell microcapsule. The at least one core-shell microcapsule comprises a core comprising at least one benefit agent and a shell surrounding the core. The shell comprises a hydrated polymer phase and a polymeric stabilizer at an interface between the shell and the core. Disclosed are also a method for preparing such an encapsulated composition and a use of such a composition to enhance the performance of a benefit agent in a consumer product.

The present invention relates to an encapsulated composition comprising at least one core-shell microcapsule, to a method for preparing such an encapsulated composition, as well as to a use of such an encapsulated composition to enhance the performance of a benefit agent in a consumer product.

It is known to incorporate encapsulated benefit agents in consumer products, such as household care, personal care and fabric care products. Benefit agents include for example fragrances, cosmetic agents, food ingredients, nutraceuticals, drugs and substrate enhancers.

Microcapsules that are particularly suitable for delivery of such benefit agents are core-shell microcapsules, wherein the core usually comprises the benefit agent and the shell is impervious or at least partially impervious to the benefit agent. Generally, these microcapsules are employed in aqueous media and the encapsulated benefit agents are hydrophobic. A broad selection of shell materials can be used, provided the shell material is impervious or at least partially impervious to the encapsulated benefit agent.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, or also fabrics or hard household surfaces in case of perfume ingredients. They can also act as a means of controlling the spatio-temporal release of a benefit agent.

A wide variety of encapsulating media as well as benefit agents suitable for the preparation of encapsulated compositions has been proposed in the prior art. Such encapsulating media include synthetic resins made from polyamides, polyureas, polyurethanes, polyacrylates, melamine-derived resins, or mixtures thereof.

However, nowadays consumers are increasingly concerned about using materials obtained from non-renewable sources, such as synthetic petrochemicals. In other words, consumers tend to favor materials the origin of which is more sustainable in terms of environment and resource protection. Nevertheless, it is generally difficult to use natural materials or materials derived from nature to address all aspects of benefit agent encapsulation.

WO 2020/233887 A1 discloses core-shell microcapsules comprising a pectin-based polymeric stabilizer and hydoxyethylcellulose. These microcapsules are bio-sourced, while still showing optimal performance in terms of stability and perfume release. However, the microcapsules still suffer from a limited ability to depose and adhere on fabrics. Furthermore, although those capsules show good biodegradability, it would be desirable to improve their capability for biodegradation.

It is therefore a problem underlying the present invention to overcome the above-mentioned shortcomings in the prior art. In particular, it is a problem underlying the present invention to enhance the deposition and adherence of capsules on substrates, more particularly on fabrics. Furthermore, the encapsulated composition should still fulfill highest standards with regard to sustainability, in particular by comprising high levels of natural materials or materials derived from nature, or even showing improved biodegradation, whilst keeping the desired benefit-agent release properties, during manufacture, storage and in application. Moreover, the compositions should be producible in an operationally safe, robust and cost-efficient process.

These problems are solved by an encapsulated composition according to the present invention. Such a composition comprises at least one core-shell microcapsule. The at least one core-shell microcapsule comprises a core comprising at least one benefit agent and a shell surrounding the core. The shell comprises a hydrated polymer phase and a polymeric stabilizer at an interface between the shell and the core.

In such an arrangement, the polymeric stabilizer provides an impervious encapsulating material, whereas the hydrated polymer phase provides the desired deposition and adherence to the substrate. Furthermore, without being bound by any theory, it is surmised that the hydrated polymer phase also provides an optimal point of attack for microbial degradation.

The polymeric stabilizer may be selected from a broad range of film-forming materials and resins. Preferably, the polymeric stabilizer is highly cross-linked, in order to decrease significantly the diffusion of the encapsulated benefit agent through the shell. Preferably the imperviousness of the shell is sufficiently high to significantly prevent the leakage of the benefit agent in extractive base, such as consumer products comprising surfactants.

In the context of the present invention, the leakage is considered as significantly reduced if the amount of the benefit agent that has leached in a consumer product base within a period of 3 months at 40° C. is less than 75 wt.-%, preferably less than 50 wt.-%, more preferably less than 25 wt.-%, and still more preferably less than 10 wt.-% of the nominal amount of encapsulated benefit agent.

In preferred embodiments of the present invention, the polymeric stabilizer is a thermosetting resin.

Thermosetting resins are typically obtained by reacting polyfunctional monomers, such as amines, isocyanates, alcohols or phenols, chlorocarboxylic acids, (meth)acrylates, epoxides, silanes and aldehydes.

In a particularly preferred embodiment of the present invention, the polymeric stabilizer is formed by reaction of an aminosilane with a polyfunctional isocyanate. Such a polymeric stabilizer has the advantage of being highly crosslinked and susceptible of providing surface anchoring groups that can be used to immobilize additional materials to complete shell formation. These additional materials may comprise additional encapsulating materials, coatings and, as described in more details hereinafter, simple and complex coacervate, and hydrogels.

The aminosilane employed in the formation of the polymeric stabilizer can be selected from a compound of Formula (I).

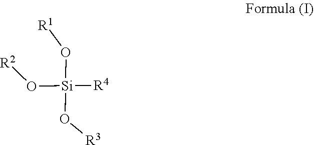

In the above Formula (I), R¹, R² and R³ are each independently C₁-C₄ linear or branched alkyl or alkenyl residues, in particular methyl or ethyl, and R⁴ is a C₁-C₁₂, preferably a C₁-C₄, linear or branched alkyl or alkenyl residue comprising an amino functional group, in particular a primary, secondary or tertiary amine.

When the functional group is a primary amine, it can be a terminal primary amine. R⁴ is then preferably a C₁-C₈, even more preferably a C₁-C₄, linear terminal primary aminoalkyl residue. Specific aminosilanes of this category are selected from the group consisting of aminomethyltriethoxysilane, 2-aminoethyltriethoxysilane, 3-aminopropyltriethoxysilane, 4-aminobutyltri-ethoxysilane, 5-aminopentyltriethoxysilane, 6-aminohexyltriethoxysilane, 7-aminohptyltriethoxysilane and 8-aminooctyltriethoxysilane.

Without being bound by any theory, it is surmised that the silane groups polycondensate with one another to form a silica network at a liquid-liquid interface that additionally stabilizes this interface.

In particular embodiments of the present invention, the aminosilane is a bipodal aminosilane. By “bipodal aminosilane” is meant a molecule comprising at least one amino group and two residues, each of these residues bearing at least one alkoxysilane moiety. The at least one bipodal aminosilane can have the Formula (II).

(O—R⁴)_{(3-f)}(R³)_{f}Si—R²—X—R²—Si(O—R⁴)_{(3-f)}(R³)_{f} Formula (II)

In the above Formula (II), X stands for —NR¹—, —NR¹—CH₂—NR¹—, —NR¹—CH₂—CH₂—NR¹—, —NR¹—CO—NR¹—, or

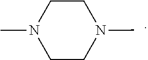

In the above Formula (II), R¹ each independently stand for H, CH₃ or C₂H₅. R² each independently stand for a linear or branched alkylene group with 1 to 6 carbon atoms. R³ each independently stand for a linear or branched alkyl group with 1 to 4 carbon atoms. R⁴ each independently stand for H or for a linear or branched alkyl group with 1 to 4 carbon atoms. f stands for 0, 1 or 2.

Bipodal aminosilanes are particularly advantageous for forming stable oil-water interfaces.

Examples of bipodal aminosilanes include, but are not limited to, bis(3-(triethoxysilyl)propyl)amine, N, N′-bis(3-(trimethoxysilyl)propyl)urea, bis(3-(methyldiethoxysilyl)propyl)amine, N,N′-bis(3-(trimethoxysilyl)propyl)ethane-1,2-diamine, bis(3-(methyldimethoxysilyl) propyl)-N-methylamine and N,N′-bis(3-(triethoxysilyl)propyl)piperazine.

The bipodal aminosilane can be a secondary aminosilane. Using a secondary bipodal aminosilane instead of primary aminosilane decreases the reactivity of the polymeric stabilizer with respect to electrophilic species, in particular aldehydes. Hence, benefit agents containing high levels of aldehydes may be encapsulated with a lower propensity for adverse interactions between core-forming and shell-forming materials.

The secondary bipodal aminosilane can be bis(3-(triethoxysilyl)propyl)amine. This particular secondary aminosilane has the advantage of releasing ethanol instead of, for instance, more toxic and less desirable methanol during the polycondensation of the ethoxysilane groups.

Other aminosilanes may also be used in combination with the aforementioned bipodal aminosilanes, in particular the aminosilanes described hereinabove.

The polyfunctional isocyanate may be selected from organic isocyanates, in which an isocyanate group is bonded to an organic residue (R—N═C═O or R—NCO). In the context of the present invention, the polyfunctional isocyanate may be selected from alkyl, alicyclic, aromatic and alkylaromatic, as well as anionically modified polyfunctional isocyanates, with two or more (e.g. 3, 4, 5, etc.) isocyanate groups in a molecule.

Preferably, the polyfunctional isocyanate is an aromatic or an alkylaromatic isocyanate, the alkylaromatic polyfunctional isocyanate having preferably methylisocyanate groups attached to an aromatic ring. Both aromatic and methylisocyanate-substituted aromatic polyfunctional isocyanates have a superior reactivity compared to alkyl and alicyclic polyfunctional isocyanates. Among these, 2-ethylpropane-1,2,3-triyl tris((3-(isocyanatomethyl)phenyl)carbamate) is particularly preferred, because of its trifunctional nature that favors the formation of intermolecular cross-links and because of its intermediate reactivity that favors network homogeneity. This alkylaromatic polyfunctional isocyanate is commercially available under the trademark Takenate D-100 N, sold by Mitsui or under the trademark Desmodur® Quix175, sold by Covestro.

As an alternative to aromatic or alkylaromatic polyfunctional isocyanates, it may also be advantageous to add an anionically modified polyfunctional isocyanates, because of the ability of such polyfunctional isocyanates to react at the oil/water interface and even in the water phase close to the oil/water interface. A particularly suitable anionically modified polyfunctional isocyanate has Formula (III).

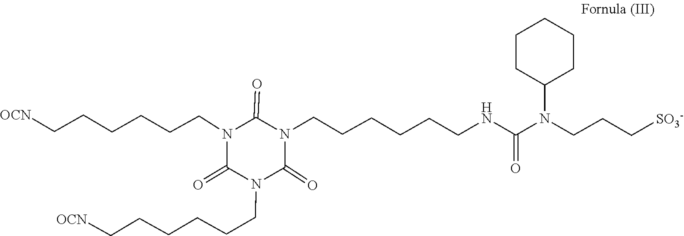

Formula (III) shows a commercially available anionically modified polyisocyanate, which is a modified isocyanurate of hexamethylene diisocyanate, sold by Covestro under the trademark Bayhydur® XP2547.

In a preferred embodiment of the present invention, polyfunctional isocyanate is 2-ethylpropane-1,2,3-triyl tris((3-(isocyanatomethyl)phenyl)carbamate).

Particularly preferably, the polymeric stabilizer is formed by reaction of bis(3-(triethoxysilyl)propyl)amine and 2-ethylpropane-1,2,3-triyl tris((3-(isocyanatomethyl)phenyl)carbamate). The combination of this particular bipodal secondary aminosilane and polyfunctional isocyanate provides advantageous interface stability and release properties. The stabilized interface is sufficiently impervious to effectively encapsulate the at least one benefit agent comprised in the core and possesses the desired surface functional groups.

In preferred embodiments of the present invention the hydrated polymer phase can be a coacervate, in particular a complex coacervate.

By “coacervate” it is meant polyelectrolyte-rich droplets coexisting with an aqueous, polyelectrolyte poor continuous phase. The droplets can agglomerate at interfaces to form an interfacial layer. In the present context, the coacervate droplets agglomerate at the interface between the polymeric stabilizer and the aqueous phase. As a result, an encapsulated composition in water is formed, comprising a plurality of core composition droplets, stabilized by the polymeric stabilizer, each droplet being surrounded by coacervate droplets.

By “complex coacervation” is meant the formation of an interfacial layer comprising a mixture of polyelectrolytes.

The phenomenon of coacervation may be observed under a light microscope, wherein it is marked by the appearance of a ring around the core composition droplet. This ring consists of the aforementioned polyelectrolyte-rich phase that has a different refractive index than the surrounding aqueous phase.

The coacervation of a polyelectrolyte is generally induced by bringing the polyelectrolyte to its isoelectric point, meaning the point where the net charge of the polyelectrolyte is zero or close to zero. This may be achieved by changing the salt concentration or the pH of the medium. In a complex coacervation, complexation occurs at the pH where one of the polyelectrolytes has an overall positive electrical charge (polycation), whereas the other polyelectrolyte has an overall negative charge (polyanion), so that the overall electrical charge of the complex is neutral.

In preferred embodiments of the present invention, the coacervate may be formed from a polycation and a polyanion.

Preferably, the pH is used as parameter driving the coacervation. Thus, the polycation preferably has a pH-dependent electrical charge. This is the case for polymers bearing primary, secondary and tertiary amino groups, such as polyamines, for example chitosan, and most proteins, for example gelatin. Proteins have the additional advantage of being prone to temperature-dependent structural transitions that may also be used to control the morphology of the coacervates. In particular, varying the temperature of some proteins may induce the formation of secondary, tertiary or quaternary structures of the protein that may also be used to control the properties of the coacervate.

Chitosan has the advantage of being derived from chitin, which is a natural polymer.

In preferred embodiments of the present invention, the polycation is selected from the group consisting of proteins and chitosan.

More particularly, the polycation can be a protein selected from the group consisting of gelatin, casein, albumin, polylysine, soy proteins, pea proteins, rice proteins and hemp proteins.

In particularly preferred embodiments of the present invention, the at least one protein is a gelatin, even more preferably a Type B gelatin.

Type B gelatin can be obtained from the alkaline treatment of collagen and is well known for its ability to form complexes with anionic polyelectrolytes, such as negatively charged polysaccharides under mild acidic conditions.

Gelatin is usually characterized by the so-called “Bloom Strength”. In the context of the present invention, the Bloom Strength refers to the rigidity of a gelatin film, as measured by so-called “Bloom Gelometer”, according to the Official Procedures of the Gelatin Manufacturers Institute of America, Inc., revised 2019, Chapter 2.1. According to this procedure, the Bloom Strength, expressed in Bloom, is equal to the weight, expressed in g, required to move vertically a standardized plunger, having a diameter of 12.5 mm, to a depth of 4 mm into a gelatin gel, which has been prepared under controlled conditions, i.e. by dissolving 6.67 wt.-% of gelatin in deionized water at 60° C., in a standardized jar, and letting the gel form for 17 hours at 10° C. The higher the weight is, the higher is the Bloom Strength of the gelatin used for making the tested gel.

In preferred embodiments of the present invention, the Type B gelatin has a Bloom Strength of 90 to 250 Bloom.

If the Bloom Strength is too low, the gel is mechanically weak and coacervates obtained therefrom may not form a self-standing layer of gelatin-rich phase around the core composition. If the Bloom Strength is too high, then the coacervates and the gelatin-rich phase obtained therefrom may be too brittle.

In preferred embodiments of the present invention, the Type B gelatin is obtainable from fish, because fish gelatin meets better acceptance within consumer than beef or pork gelatin, mainly due to health concerns, sociological context or religious rules.

Alternatively, the protein may be a vegetable protein, in particular a pea protein and/or a soy protein, which have the advantage of being vegan.

The polycation may be a denaturated protein. In the contrary to native proteins, denaturated proteins have been deprived from their ability to form secondary, tertiary or quaternary structures and are essentially amorphous. Such amorphous proteins may form more impervious films compared to native proteins and therefore also contribute to the encapsulating power of the shell. Denaturation may be achieved by treating the protein with chemical or physical means, such as acid or alkaline treatment, heat or exposure to hydrogen bond disrupting agents.

In cases where the polycation is chitosan, the chitosan can have a molecular weight between 3′000 and 1′000′000 g/mol, more particularly between 10′000 and 500′000 g/mol, still more particularly between 30′000 and 300′000 g/mol.

In one embodiment, the polycation is a permanently charged cationic polysaccharide, such as cationic hydroxypropyltrimonium starch or hydroxypropyltrimonium guar gum. Such cationic polysaccharides are of vegetal origin.

The polyanion may be any negatively charged polymer. However, as the pH is preferably used to control coacervation, it may be more advantageous that the electrical charge of the polymer is pH-dependent. Such polymer may be selected from polymers having pendent carboxylic groups, such as methacrylic acid and acrylic acid polymers and copolymers, hydrolyzed maleic anhydride copolymers and polysaccharides bearing carboxylic groups.

In preferred embodiments of the present invention, the polyanion is a polysaccharide comprising carboxylate groups and/or sulfate groups.

Polysaccharides comprising carboxylate groups are particularly suitable for complex coacervation with proteins. This is due to the fact that the net electrical charge of these polysaccharides may be adjusted by adjusting the pH, so that the complexation with ampholytic proteins is facilitated. Complexation occurs at the pH where the protein has an overall positive electrical charge, whereas the polysaccharide as an overall negative charge, so that the overall electrical charge of the complex is neutral. These polysaccharides include native polysaccharides, i.e. unmodified from nature, and modified polysaccharides.

The polysaccharide comprising carboxylic acid groups may comprise uronic acid units, in particular hexuronic acid units. Such polysaccharides are broadly available in nature.

The hexuronic acid units can be selected from the group consisting of galacturonic acid units, glucuronic acid units, in particular 4-O-methyl-glucuronic acid units, guluronic acid units and mannuronic acid units.

The polysaccharide comprising carboxylic acid groups may be branched. Branched polysaccharides comprising carboxylic acid groups have the advantage of forming more compact networks than linear polysaccharides and therefore may favor the imperviousness of the encapsulating shell, resulting in reduced leakage and greater encapsulation efficiency.

The carboxylate groups can be at least partially present in the form of the corresponding carboxylate salt, in particular the corresponding sodium, potassium, magnesium or calcium carboxylate salt.

In particular embodiments of the present invention, the polyanion is selected from the group consisting of pectin, gum arabic and alginate.

Among the pectins, the carboxylic acid groups can be partially present in the form of the corresponding methyl ester. The percentage of carboxylic acid groups that are present in the form of the corresponding methyl ester can be from 3% to 95%, preferably from 4% to 75%, more preferably from 5 to 50%. Pectins comprising carboxylic groups, of which 50% or more are present in the form of the corresponding methyl ester, are referred to as “high methoxylated”. Pectins comprising carboxylic acid groups, of which less than 50% are present in the form of the corresponding methyl ester, are referred to as “low methoxylated”.

Among the two variants of gum Arabic, i.e. gum acacia Senegal and gum acacia Seyal, gum acacia Senegal is preferred, owing to the higher level of glucuronic acid in gum acacia Senegal.

The hydrated polymer phase can be a hydrogel.

In context of the present invention, a “hydrogel” is a three-dimensional (3D) network of hydrophilic polymers that can swell in water, while maintaining the structure due to chemical or physical cross-linking of individual polymer chains.

Such a hydrogel can be formed by several methods at interfaces, especially by self-assembly of polyelectrolytes around existing interfaces, covalent grafting of pre-formed hydrogel particles in solution, polymerization of hydrosoluble monomers initiated at the interface and phase separation of water soluble macromolecules onto the interface.

To avoid any ambiguity, in context of the present invention, a coacervate, especially a complex coacervate, which is cross-liked, in particular by covalent bonds, is considered as a hydrogel.

The applicant has found that the use of hydrogels particularly enhances both the deposition and adherence of microcapsules on substrates, in particular on fabrics.

The hydrogel can be interlinked with the polymeric stabilizer, in particular via the functional groups present on the surface of this stabilizer.

This allows the locking of the hydrogel layer onto the polymeric stabilizer present at droplet interface, making the shell composed of a polymer composite, instead of only a blend.

Both hydrogel cross-linking and hydrogel interlinking with the polymeric stabilizer may be performed sequentially or simultaneously.

In preferred embodiments of the present invention, the hydrogel is a crosslinked coacervate, in particular a complex coacervate crosslinked with polyfunctional aldehyde, more particularly a difunctional aldehyde selected from the group consisting of succinaldehyde, glutaraldehyde, glyoxal, benzene-1,2-dialdehyde, benzene-1,3-dialdehyde, benzene-1,4-dialdehyde, piperazine-N,N-dialdehyde and 2,2′-bipyridyl-5,5′-dialdehyde. Difunctional aldehydes are known to be effective cross-linking agents for proteins.

The hydrogel can be thermosensitive and possess a gelation temperature, in particular between 20° C. and 50° C., preferably between 25° C. and 40° C. When using such a hydrogel, the deposition performance of the capsules on fabic can increase, when washing the fabric at a temperature which is above hydrogel gelation temperature.

The shell can be further stabilized with a stabilizing agent. Preferably the stabilizing agent comprises at least two carboxylic acid groups. Even more preferably, the stabilizing agent is selected from the group consisting of citric acid, benzene-1,3,5-tricarboxylic acid, benzene-1,2,4-tricarboxylic acid, 2,5-furandicarboxylic acid, itaconic acid, poly(itaconic acid) and combinations thereof.

A further aspect of the present invention relates to a method for preparing an encapsulated composition, in particular an encapsulated composition as described herein above. This method comprises the steps of:
a) Providing an aqueous phase;
b) Providing an oil phase comprising at least one benefit agent;
c) Emulsifying the oil phase in the aqueous phase to form an emulsion of oil droplets in the aqueous phase;
d) Forming a polymeric stabilizer surrounding the oil droplets;
e) Providing a hydrated polymer phase on an outer face of the polymeric stabilizer, in order to obtain a microcapsule shell.

Emulsions of oil droplets in an aqueous phase have the advantage of providing a plurality of droplets that may be used as template for shell formation, wherein the shell is built around each of these droplets. Additionally, the droplet size distribution may be controlled in emulsions, by controlling the conditions of emulsifications, such as stirring speed and stirrer geometry. As a result, a plurality of microcapsules is obtained with controlled average size and size distribution, wherein the oil phase is encapsulated and forms thereby the core of the microcapsules. The appropriate stirring speed and geometry of the mixer can be selected in order to obtain the desired average droplet size and droplet size distribution.

In a process according to the present invention, a one-liter vessel equipped with a turbine, or a cross-beam stirrer with pitched beam, such as a Mig stirrer, and having a stirrer diameter to reactor diameter of 0.6 to 0.8 may be used. Microcapsules can be formed in such reactor having a volume average size (d50) of 30 μm or less, more particularly 20 μm or less, at a stirring speed from about 100 to about 1200 rpm, more particularly from about 600 to 1000 rpm. Preferably, a Mig stirrer is used operating at a speed of 850+/−50 rpm. The person skilled in the art will however easily understand that such stirring conditions may change depending on the size of the reactor and of the batch size, on the exact geometry of the stirrer on the ratio of the diameter of the stirrer to the diameter of the reactor diameter ratios. For example, for a Mig stirrer with stirrer to reactor diameter ratio from 0.5 to 0.9 and slurry volumes ranging from 0.5 to 8 tons, the preferable agitation speed in the context of the present invention is from 150 rpm to 50 rpm.

With respect to step c), the emulsification of the oil phase in the aqueous phase may be conducted in presence of a polymeric surfactant. The polymeric surfactant helps to promote the formation of dispersed oil droplets with desirable droplet size.

Polymeric surfactants that can be used for the sake of the present invention are well known to the person skilled in the art and include a broad range of hydrocolloids, such as copolymers of acrylamide, benzene sulphonated, (meth)acrylic acid, maleic anhydride, polyvinyl alcohol, polyvinyl pyrrolidone, and native of modified biopolymers, such as proteins, lignins and polysaccharides.

With respect to step d), the polymeric stabilizer may be formed by reaction of an aminosilane with a polyfunctional isocyanate, which are preferably both comprised in the oil phase provided in step b).

The polymeric stabilizer may additionally be formed by combination of the aminosilane with the polymeric surfactant.

With respect with step e), the hydrated polymer phase may be formed as a complex coacervate from a polycation and a polyanion.

In preferred embodiments of the present invention, the polyanion employed in step e) for formation of the complex coacervate is identical to the polymeric surfactant employed in step c) for emulsification of the oil phase in the aqueous phase.

By using one component of the system for a dual purpose, the overall complexity and cost of the encapsulated composition can be reduced. Furthermore, the environmental impact of the product can be improved, as fewer materials are needed for its manufacture.

Apart from the above advantages, in cases where the polymeric surfactant is anchored to or comprised in the polymeric stabilizer, in particular by combination of the polymeric surfactant with an aminosilane, formation of a composite structure between the polymeric stabilizer and the complex coacervate is possible. This also allow reduce “free” polymeric water soluble residues in the continuous phase.

As mentioned herein above, the polycation can be selected from the group consisting of proteins and chitosan.

In particularly preferred embodiments of the present invention, the polymeric surfactant and/or the polyanion is a polysaccharide comprising carboxylate and/or sulfonate groups.

Carboxylate and/or sulfonate groups provide strong electrostatic interactions, but at the same time avoid extensive dehydration. Hydrated polymer phases formed by interactions between these groups stay well hydrated (a contrario to precipitates) and possess better viscoelastic properties, leading to improved deposition.

The method according to the present invention may additionally comprise the following step:
f) Crosslinking the complex coacervate is to form a hydrogel, optionally also interlinking the hydrogel with the polymeric stabilizer.

After formation of the microcapsules, the encapsulated composition is usually cooled to room temperature. Before, during or after cooling, the encapsulated composition may be further processed. Further processing may include treatment of the composition with anti-microbial preservatives, which preservatives are well known in the art. Further processing may also include the addition of a suspending aid, such as a hydrocolloid suspending aid to assist in the stable physical dispersion of the microcapsules and prevent any creaming or coalescence. Any additional adjuvants conventional in the art may also be added during further-processing.

The benefit agent comprised in the core can be selected from the group consisting of fragrance ingredients, cosmetic ingredients and biologically active ingredients.

In particular embodiments of the present invention, the core comprises at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated perfumes according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy) butan-2-olAMBERKETAL (3,8,8,11a-tetramethyldodecahydro-1H-3,5a-epoxynaphtho[2,1-c]oxepine); AMBERMAX (1,3,4,5,6,7-hexahydro-.beta., 1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((25,4S)-1,7,7-trimethylbicyclo[2.2.1]heptan-2-yl acetate); BOURGEONAL (3-(4-(tert-butyl)phenyl)propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl) quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4 (5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl) hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl)acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-CYCLOMYRAL (8,8-dimethyl-1,2,3,4,5,6,7,8-hydroxybenzoate); octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA (1-(2,6,6-trimethyl-1-cyclohex-3-enyl)but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE DIHYDRO (3-methyl-2-pentylcyclopent-2-enone); MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5 (6H)-ylidene) butanal); EBANOL ((E)-3-methyl-5-(2,2,3-trimethylcyclopent-3-en-1-yl) pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl)ethanone); FLORALOZONE (3-(4-ethylphenyl)-2,2-dimethylpropanal); FLORHYDRAL (3-(3-isopropylphenyl)butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl) pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-3-oxo-2-pentylcyclopentaneacetate); 12-en-2-one); HEDIONE (methyl HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl)phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl) methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy)hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl) propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL (2-(6,6-dimethylbicyclo[3.1.1]hept-2-en-2-yl)ethyl acetate); ACETATE NYMPHEAL (3-(4-(2-methylpropyl)-2-methylphenyl)propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl)butanamide); PEACH PURE (5-heptyldihydrofuran-2 (3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl) acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl) propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2 (3H)-one); RADJANOL SUPER ((E)-2-ethyl-4-(2,2,3-trimethylcyclopent-3-en-1-yl)but-2-en-1-ol); RASPBERRY KETONE (4-(4-hydroxyphenyl)butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE (3-methyl-5-(2,2,3-trimethylcyclopent-3-en-1-yl) pentan-2-ol); EXTRA SCENTAURUS CLEAN (ethyl (Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl)ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl) pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl) propan-2-ol) TERPINEOL ALPHA (2-(4-methyl-1-cyclohex-3-enyl) propan-2-ol); TERPINEOL PURE (2-(4-methylcyclohex-3-en-1-yl) propan-2-ol); TERPINOLENE (1-methyl-4-(propan-2-ylidene)cyclohex-1-ene); TERPINYL ACETATE (2-(4-methyl-1-cyclohex-3-enyl) propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In particularly preferred embodiments of the present invention, more than 75 wt.-%, preferably more than 80 wt.-%, even more preferably more than 85 wt.-%, even still more preferably more than 90 wt.-%, even yet still more preferably more than 95 wt.-%, of the fragrance ingredients are biodegradable and selected from ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((25,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl) pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl)butanal); FLORIDILE ((E)-undec-9-enenitrile); GALBANONE PURE (1-(5,5-dimethylcyclohex-1-en-1-yl) pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); ISOAMYL ACETATE (3-methylbutyl acetate); ISOAMYL BUTYRATE (3-methylbutyl butanoate); ISOEUGENOL ((E)-2-methoxy-4-(prop-1-en-1-yl)phenol); ISOJASMONE B 11 (2-hexylcyclopent-2-en-1-one); ISORALDEINE ((E)-3-methyl-4-(2,6,6-trimethylcyclohex-2-en-1-yl)but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl)phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl) propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl)propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2 (3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2 (3H)-one); RASPBERRY KETONE (4-(4-hydroxyphenyl)butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl) propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These ingredients have the advantage of providing microcapsules which are particularly sustainable.

In context of the present invention, a “biodegradable ingredient”, or a biodegradable material in general, for instance a shell material, is a material which meets the pass criteria for “inherently biodegradable” and/or “readily biodegradable” in at least one OECD biodegradation study. In order to avoid any ambiguity, this means that if an ingredient passes one test but fails one or more other ones, the pass result overrules the other test results.

For assessment of the pass criteria for “readily biodegradable”, the biodegradation study can be selected from the group consisting of OECD Method 301B, OECD Method 301C, OECD Method 301D, OECD Method 301F and OECD Method 310.

OECD Method 301B, OECD Method 301C, OECD Method 301D and OECD Method 301F are described in the OECD Guidelines for the Testing of Chemicals, Section 3, Test No. 301: Ready Biodegradability (Adopted: 17Jul. 1992; https://doi.org/10.1787/97892640703-(9-en).

OECD Method 310 is described in the OECD Guidelines for the Testing of Chemicals, Section 3, Test No. 310: Ready Biodegradability-CO2 in sealed vessels (Headspace Test) (Adopted: 23 Mar. 2006; Corrected: 26 Sep. 2014; https://doi.org/10.1787/978926-(016316-en).

In a particular aspect of the present invention, the pass criteria for “readily biodegradable” are assessed according to OECD Method 301F, which refers to manometric respirometry. In this method the pass level for “ready biodegradability” is to reach 60% of theoretical oxygen demand and/or chemical oxygen demand. This pass value has to be reached in a 10-day window within the 28-day period of the test. The 10-day window begins when the degree of biodegradation has reached 10% of theoretical oxygen demand and/or chemical oxygen demand and must end before day 28 of the test.

Given a positive result in a test of ready biodegradability, it may be assumed that the chemical will undergo rapid and ultimate biodegradation in the environment (Introduction to the OECD Guidelines for the Testing of Chemicals, Section 3, Part 1: Principles and Strategies Related to the Testing of Degradation of Organic Chemicals; Adopted: July 2003).

For assessment of the pass criteria for “inherently biodegradable”, the biodegradation study can be OECD Method 302C, but also OECD Method 301F can be used, although with different pass criteria. Also these methods are suitable for volatile materials.

OECD Method 302C is described in the OECD Guidelines for the Testing of Chemicals, Section 3, Test No. 302C: Inherent Biodegradability: Modified MITI Test (II) (Adopted: 12 May 1981; Corrected 8 Sep. 2009; https://doi.org/10.1787/9789264070400-en).

In a particular aspect of the present invention, the pass criteria for “inherently biodegradable” are assessed by OECD Method 302C. In this method the pass level for “inherently biodegradability” is then to reach 70% of theoretical oxygen demand. There is no time limit to reach this level.

Biodegradation rates above 70% may be regarded as evidence of inherent, ultimate biodegradability (OECD Guidelines for the Testing of Chemicals, Section 3, Part 1: Principles and Strategies Related to the Testing of Degradation of Organic Chemicals; Adopted: July 2003).

If OECD Method 301F is used for assessment of the pass criteria for “inherently biodegradable”, the pass level is 60% of theoretical oxygen demand and/or chemical oxygen demand. This pass value can be reached after the 28-day period of the test, which is usually extended to 60 days. No 10-day window applies.

In the present context, if an ingredient is an essential oil, it is considered to be a “biodegradable ingredient” if all of its constituents present at a level ≥1 wt.-% fall under the definition of “inherently biodegradable” and/or “readily biodegradable” as defined herein above. However, the essential oil can also be subjected to the above-mentioned biodegradation tests.

The core composition may also comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

The core composition may also comprise at least one functional cosmetic ingredient. The functional cosmetic ingredients for use in the encapsulated composition are preferably hydrophobic. Preferably, the cosmetic ingredients have a calculated octanol/water partition coefficient (ClogP) of 1.5 or more, more preferably 3 or more. Alternatively preferred, the ClogP of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Particularly useful functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C₁₈-Sphingosine, CAS-No: 100403-19-8).

In order to offer an optimal balance between stability, deposition on substrate and performance, the volume average size (d50) of the microcapsuels can be from 1 to 50 μm, preferably from 5 to 30 μm, even more preferably from 7 to 20 μm. Microcapsules having diameters smaller than 5 μm show large surface to volume ratios and are therefore more prone to leaching, whereas, as the number of microcapsule decreases with increasing diameter, too large microcapsules may not be numerous enough to provide noticeable benefits. Furthermore, large microcapsules may be visible in the product or let visible stain on the substrate.

The method according to the present invention may comprise the additional step of drying the microcapsules, in order to obtain a microcapsule power.

Optionally, additional materials may be added to this powder, such as carrier materials, such as salts, silicates, clays and carbohydrates, fire proofing materials, additional functional materials, such as fragrance ingredients, cosmetic ingredients, biologically active ingredients, and substrate enhancers, additional encapsulating materials, such as polysaccharides, proteins, alkoxysilanes, synthetic polymers and copolymers, surfactants and waxes.

Drying methods such as spray-drying, spray-coating, belt and drum drying may be employed. These methods are well known to the art.

In particular, the drying process may be accompanied by an additional encapsulation process, wherein an additional functional material is entrapped in an additional encapsulating material. For example, the slurry to be dried may comprise, additionally to the core-shell microcapsules obtained in the process according to the present invention, at least one non-encapsulated functional material and at least one water-soluble encapsulating material, so that the functional material, that is not encapsulated in the core-shell microcapsule, is entrapped in the water-soluble encapsulating material during drying. Typically, the at least one water-soluble encapsulating material comprises at least one hydrocolloid, such as starch octenyl succinate and gum acacia. The hydrocolloid promotes and stabilizes the dispersion of the non-encapsulated material in the aqueous phase of the slurry, so that, upon drying, a matrix is formed around or coexisting with the core-shell microcapsules.

The functional material that is encapsulated in the core-shell microcapsules may comprise a first fragrance, whereas the functional material entrapped in the water-soluble encapsulating material may comprise a second fragrance, wherein the first and second fragrances are identical or different.

Combining at least two encapsulation processes has the advantage of providing different mechanisms for releasing the functional material, for example a combination of moisture-induced and mechanical stress-induced releases.

The drying step may also be accompanied or followed by mechanical or thermal treatment, such as spheronization, granulation and extrusion.

In another aspect, the present invention relates to an encapsulated composition obtainable by a method as described hereinabove.

The encapsulated composition may be in the form of liquid slurries, powder, granulates, flakes or extrudates. The composition may be used as such, for example as fragrance booster, or in diluted form in a product.

Encapsulated compositions in the form of liquid slurries may comprise from 10 to 50 wt.-%, more particularly from 15 to 25 wt.-%, of core-shell microcapsules.

Encapsulated compositions in solid form may comprise from 1 to 100 wt.-% of core-shell microcapsules. However, depending on the application or on the nature of the functional material, it may be preferable to limit or, in the contrary, to maximize the level of core-shell microcapsules in the solid form. For example, a limitation of the level of the core-shell microcapsules in the solid may be particularly desired if the encapsulated material is flammable, reactive, pungent or expensive.

Hence, the optimal level of encapsulated fragrance ingredients in a solid composition may be less than 50 wt.-%, more particularly less than 35 wt.-% and still more particularly less than 20 wt.-%, or even less than 15 wt.-%, depending on the flammability of such fragrance ingredients and the associated explosion risks.

The encapsulated fragrance may be diluted in a carrier material mentioned herein above.

In yet another aspect, the present invention relates to a use of an encapsulated composition as described herein above to enhance the performance of a benefit agent in a consumer product.

The present invention also relates to a consumer product comprising an encapsulated composition as described herein above. The consumer product is preferably selected from the group consisting of fabric care detergents and conditioners, hair care conditioners, shampoos, heavy duty liquid detergents, hard surface cleaners, detergent powders, soaps, shower gels and skin care products.

Encapsulated compositions according to the present invention are particularly useful when employed as perfume delivery vehicles in consumer goods that require, for delivering optimal perfumery benefits, that the microcapsules adhere well to a substrate on which they are applied. Such consumer goods include hair shampoos and conditioners, as well as textile-treatment products, such as laundry detergents and conditioners.

Particular features and further advantages of the present invention become apparent from the following examples.

### Example 1—Formation of Microcapsules Comprising a Hydrogel Formed by the Combination of Pectin and Gelatin (According to the Present Invention)

The microcapsules were obtained as follows:
a) A core composition was prepared by admixing 0.7 g of bipodal aminosilane (bis(3-triethoxysilylpropyl)amine), 0.48 g Takenate D-110N (ex Mitsui) and 38.5 g of fragrance composition;
b) The core composition obtained in step a) was emulsified in a mixture of 1.0 g high methoxylated grade pectin (of type APA 104, ex Roeper) in 73.3 g of water by using a 300 ml reactor and a cross-beam stirrer with pitched beam operating at a stirring speed of 600 rpm at a temperature of 25+/−2° C. for 10 min;
c) The temperature of the system was raised to 85+/−2° C. over 4 hours, 0.3 g of trimesic acid (1,3,5-benzenetricarboxylic acid) were added and the system was maintained at this temperature for 1.3 h while maintaining stirring as in step b);
d) The system was slowly cooled to 40° C. over 2.25 hours, while maintaining stirring as in step b);
e) At a temperature of 40+/−2° C., 10 g of a 10% gelatin solution in water was added, while maintaining stirring as in step b);
f) The system was slowly cooled to 10° C. over 2.25 hours, while maintaining stirring as in step b);
g) Once the system had reached the temperature of 10° C., 0.02 g of a 50 wt.-% glutaraldehyde solution in water was added and the system was maintained at this temperature for 1 hour, while maintaining stirring as in step b), in order to form a slurry of core-shell microcapsules;
h) The slurry of core-shell capsules obtained in step f) was finally let to stabilize at room temperature.

The solid content of the slurry obtained was 33.1 wt.-%, the volume median size (d50) of the capsules was 32 μm and the encapsulation efficiency of 99%.

### Example 2—Formation of Microcapsules Comprising Cellulosic Shell (Comparative Example)

The microcapsules were obtained as follows:
a) A core composition was prepared by admixing 0.66 g bipodal aminosilane (bis(3-triethoxysilylpropyl)amine), 0.48 g Takenate D-110N (ex Mitsui) and 38.5 g of fragrance composition;
b) The core composition obtained in step a) was emulsified in a mixture of 1.4 g high methoxylated grade pectin (of type APA 104, ex Roeper) in 66.2 g of water by using a 300 ml reactor and a cross-beam stirrer with pitched beam operating at a stirring speed of 600 rpm at a temperature of 25+/−2° C. for 10 min;
c) The temperature of the system was raised to a temperature of 85+/−2° C. over 4 hours, 0.3 g of trimesic acid (1,3,5-benzenetricarboxylic acid) and 1.8 g of hydroxyethylcellulose (Natrosol 250L, ex Ashland) where added, and this temperature was maintained for 1.30 h while maintaining stirring as in step b) to complete the formation of core-shell capsules;
d) The slurry of core-shell capsules obtained in step c) was cooled to room temperature.

The solid content of the slurry obtained was 39.0 wt.-%, the volume average size (d50) of the capsules was 20 μm and the encapsulation efficiency of 99%.

### Example 3—Formation of Microcapsules Comprising a Hydrogel Formed by the Combination of Gum Arabic and Gelatin (According to the Present Invention)

The microcapsules were obtained as follows:
a) A slurry of melamine-formaldehyde microcapsules was prepared according to the procedure described in WO 2016/207187 A1, Example 2b. This slurry was washed 2 times by centrifugation and the pH was adjusted to 4.5 with 10% HCl solution;
b) A 10 wt.-% gelatin solution in deionized water was prepared and the pH was adjusted to pH 7 with NaOH;
c) A 2 wt.-% gum Arabic solution in deionized water was prepared and the pH was adjusted to pH 7 with NaOH;
d) In a separate vessel, 50 g of the 10 wt.-% gelatin solution in water and 50 g of the 2 wt.-% gum Arabic in water were admixed and the resulting mixture heated to a temperature of 40° C.;
e) The pH of the mixture obtained in step d) was then reduced to 4.5 with 10% HCl solution under vigourous stirring (500 RPM). The temperature was maintained at 40° C. for 15 minutes and then decreased to 35° C. to induce coacervation, while maintaining the stirring.
f) The slurry obtained in step a) was then added to the mixture obtained in step e) in order to obtain a microcapsule concentration of 13 wt.-%;
i) The system was cooled to 10° C., while maintaining the stirring as in step e), 0.3 g of a 50 wt.-% of glutaraldehyde in water was added, and the system was maintained at this temperature for 1 hour while maintaining the stirring, in order to form a slurry of core-shell microcapsules;
g) The slurry of core-shell capsules obtained in step h) was finally let to stabilize at room temperature.

The solid content of the slurry obtained was 15.7 wt.-%, the volume average size (d50) of the capsules was 23 μm and the encapsulation efficiency of 99%.

### Example 4—Formation of Microcapsules Comprising an Aminoplast Resin (Comparative Example)

Melamine-formaldehyde microcapsules were prepared according to the procedure described in WO 2016/207187 A1, Example 2b.

The solid content of the slurry obtained was 40.4 wt.-%, the volume average size (d50) of the capsules was 19 μm and the encapsulation efficiency of 99%.

### Example 5-Comparison of Microcapsule Deposition on Fabrics

The deposition of the microcapsules on fabrics was determined in model washing set-up (Tergotomer). The device comprised a cylindrical stainless steel vessel equipped with a three-blade stirring device.

1.0 g of fabric conditioner, comprising 0.1 wt.-% of microcapsules containing fluorescent a dye (Uvinul A (diethylamino hydroxybenzoyl hexyl benzoate)) was added to 500 ml of tab water and dispersed in the Tergotometer at the washing temperature (for example 25° C., 30° C. or 40° C.; see results below). 20 g of cotton fabrics was added and the stirrer was set at a speed of 80 rpm for 10 minutes and then switched off. In order to mimic the rinse step, 300 ml of the liquor was removed and replaced with 800 ml of fresh water at 25° C., 30° C. or 40° C., respectively. The stirring was switched on again for 10 minutes and then switched off. The water was removed and the fabric left to dry at room temperature. The amount of deposited microcapsules was determined by fluorimetry.

### Results:

TABLE 1

| | Normalized Deposition at 25° C. | | |
|---|---|---|---|
| | (comparative example = 1) | | |
| | Laundry Care Conditioner | No Base | |
| Example 1 | 1.34 +/− 0.03 | 1.5 +/− 0.2 | — |
| Example 2 | 1.00 +/− 0.02 | 1.0 +/− 0.1 | — |
| (comparative) | | | |
| Example 3 | — | — | 2.4 +/− 0.1 |
| Example 4 | — | — | 1.0 +/− 0.1 |
| (comparative) | | | |

TABLE 2

| | Normalized Deposition at 30° C. |
|---|---|
| | (comparative example = 1) |
| | Laundry Care Conditioner |
| Example 3 | 1.43 +/− 0.1 |
| Example 4 (comparative) | 1.0 +/− 0.1 |

TABLE 3

| | Normalized Deposition at 40° C. |
|---|---|
| | (comparative example = 1) |
| | No Base |
| Example 3 | 3.3 +/− 0.1 |
| Example 4 (comparative) | 1.0 +/− 0.1 |
| Example 4 (at 25° C.) | 1.0 +/− 0.1 |

Capsules with a hydrated polymer phase showed a higher deposition at all temperatures examined and in different environments, such as water or laundry care conditioner. In laundry care conditioner the increase was between 34 and 43%, with respect to control capsules. In water, values of 50% up to 330% were achieved.

Furthermore, it was observed that capsule deposition without hydrated polymer phase does not change significantly as a function of temperature. On the other hand, for capsules with hydrated polymer phase an increase in deposition from 240 to 330% was found by going from 25° C. to 40° C.

TABLE 4

| | Biodegradation according to |
|---|---|
| | OECD Method 301F after 28 Days |
| Example 1 | >60% |
| Example 2 (comparative) | <40% |

Presence of a shell with a hydrogel (example 1) show a biodegradability of more than 60% while capsule without hydrogel (example 2) have biodegradability less than 40%.

1. An encapsulated composition comprising at least one core-shell microcapsule, wherein the at least one core-shell microcapsule comprises a core comprising at least one benefit agent and a shell surrounding the core, wherein the shell comprises a hydrated polymer phase and a polymeric stabilizer at an interface between the shell and the core.

2. The encapsulated composition according to claim 1, wherein the polymeric stabilizer is a thermosetting resin.

3. The encapsulated composition according to claim 2, wherein the polymeric stabilizer is formed by reaction of an aminosilane with a polyfunctional isocyanate.

4. The encapsulated composition according to claim 3, wherein the aminosilane is a bipodal aminosilane.

5. (canceled)

6. (canceled)

7. (canceled)

8. The encapsulated composition according to claim 1, wherein the hydrated polymer phase is a coacervate.

9. The encapsulated composition according to claim 37, wherein the complex coacervate is formed from a polycation and a polyanion.

10. The encapsulated composition according to claim 9, wherein the polycation is selected from the group consisting of proteins, chitosan and cationic polysaccharides.

11. The encapsulated composition according to claim 10, wherein the polycation is a protein selected from the group consisting of gelatin, casein, albumin, polylysine, soy proteins, pea proteins, rice proteins, hemp proteins, and combinations thereof.

12. The encapsulated composition according to claim 11, wherein the protein is a gelatin.

13. (canceled)

14. The encapsulated composition according to claim 10, wherein the polycation is a denaturated protein.

15. The encapsulated composition according to claim 10, wherein the polycation is a cationic polysaccharide selected from the group consisting of cationic hydroxypropyltrimonium starch, hydroxypropyltrimonium guar gum, and combinations thereof.

16. The encapsulated composition according to claim 9, wherein the polyanion is a polysaccharide comprising carboxylate groups and/or sulfate groups.

17. The encapsulated composition according to claim 16, wherein the polysaccharide comprising carboxylate groups comprises uronic acid units.

18. (canceled)

19. (canceled)

20. (canceled)

21. The encapsulated composition according to claim 16, wherein the polyanion is selected from the group consisting of pectin, gum arabic, alginate and combinations thereof.

22. The encapsulated composition according to claim 1, wherein the hydrated polymer phase is a hydrogel.

23. The encapsulated composition according to claim 22, wherein the hydrogel is interlinked with the polymeric stabilizer.

24. The encapsulated composition according to claim 22, wherein the hydrogel is a crosslinked coacervate.

25. A method for preparing the encapsulated composition according to claim 1, the method comprising the steps of:
a) Providing an aqueous phase;
b) Providing an oil phase comprising at least one benefit agent;
c) Emulsifying the oil phase in the aqueous phase to form an emulsion of oil droplets in the aqueous phase;
d) Forming a polymeric stabilizer surrounding the oil droplets;
e) Providing a hydrated polymer phase on an outer face of the polymeric stabilizer, in order to obtain a microcapsule shell.

26. The method according to claim 25, wherein, in step c), emulsification of the oil phase in the aqueous phase is conducted in the presence of a polymeric surfactant.

27. The method according to claim 25, wherein, in step d), the polymeric stabilizer is formed by reaction of an aminosilane with a polyfunctional isocyanate.

28. The method according to claim 27, wherein, in step d), the polymeric stabilizer is formed by combination of the aminosilane with the polymeric surfactant.

29. The method according to claim 25, wherein, in step e), the hydrated polymer phase is formed as a complex coacervate from a polycation and a polyanion.

30. The method according to claim 29, wherein the polyanion employed in step e) for formation of the complex coacervate is identical to the polymeric surfactant employed in step c) for emulsification of the oil phase in the aqueous phase.

31. A The method according to claim 29, wherein the polycation is selected from the group consisting of proteins, chitosan, and cationic polysaccharides and combinations thereof.

32. The method according to claim 26, wherein the polymeric surfactant and/or the polyanion is a polysaccharide comprising carboxylate and/or sulfonate groups.

33. The method according to claim 29, further comprising the following step:
f) Crosslinking the complex coacervate is to form a hydrogel.

34. An encapsulated composition obtained by a method according to claim 25.

35. A method of utilizing an encapsulated composition according to claim 1 to enhance the performance of a benefit agent in a consumer product.

36. A consumer product comprising an encapsulated composition according to claim 1, wherein the consumer product is selected from the group consisting of fabric care detergents and conditioners, hair care conditioners, shampoos, heavy duty liquid detergents, hard surface cleaners, detergent powders, soaps, shower gels, skin care products and combinations thereof.

37. The encapsulated composition according to claim 8, wherein the coacervate is a complex coacervate.

38. The encapsulated composition according to claim 24, wherein the crosslinked coacervate is a complex coacervate crosslinked with polyfunctional aldehyde.
